(19) United States
(12) Patent Application Publication
(10) Pub. No.: US 2026/0295240 A1
(43) Pub. Date: Oct. 1, 2026

(54) NERVE THERAPY SYSTEM AND METHOD USING LOW FREQUENCY ALTERNATING CURRENT

(71) Applicant: Purdue Research Foundation, West Lafayette, IN (US)

(72) Inventors: Kenichi Yoshida, Carmel, IN (US); Nathaniel Liam Lazorchak, Indianapolis, IN (US); Awadh Mubarak M Alhawwash, Indianapolis, IN (US)

(21) Appl. No.: 19/634,720

(22) Filed: Mar. 31, 2026

Related U.S. Application Data
(60) Provisional application No. 63/780,871, filed on Mar. 31, 2025.

(51) Int. Cl. A61N 1/05; A61N 1/36

(52) U.S. Cl. CPC A61N 1/0556; A61N 1/36157; A61N 1/36171

(57) ABSTRACT

A nerve therapy system includes a plurality of electrodes configured to be positioned adjacent a nerve. The system also includes a signal generator electrically connected to the electrodes and configured to generate a low frequency alternating current (“LFAC”) and apply the LFAC to the electrodes. The system further includes a pulse generator electrically connected to the electrodes and configured to generate a pulse current and apply the pulse current to the electrodes simultaneously with the LFAC.

### CROSS-REFERENCE TO RELATED APPLICATIONS

This application claims the benefit of U.S. Provisional Application No. 63/780,871, filed on Mar. 31, 2025. The entire disclosure of the above application is incorporated herein by reference.

### FIELD

The present technology relates generally to nerve fiber therapy systems and methods and more particularly to nerve fiber therapy systems and methods using low frequency alternating current (LFAC).

### INTRODUCTION

This section provides background information related to the present disclosure which is not necessarily prior art.

The nervous system in animals, including humans, serves to transmit signals, produced by electrochemical processes, to and from different parts of the body. The system includes a central nervous system (brain, spinal cord) and a peripheral nervous system. The peripheral nervous system consists mainly of nerves, which are cable-like bundles of nerve fibers, also known as axons. The axons are bundled together into nerve fascicles. As such, each nerve may include multiple fascicles having different sizes and functions.

Certain techniques of nerve therapy involve utilizing electrodes placed in contact with or adjacent to a nerve. An electrical current can then be applied to the electrodes. In some cases, this results in blocking of signals being transmitted through the nerve fascicles. In other cases, this may result in generating (i.e., activating) a signal to be transmitted through the nerve fascicles.

U.S. Pat. No. 11,198,005 to Yoshida et al. describes a nerve activity blocking system that applies a low frequency alternating current (LFAC) to a nerve. While this system is effective, when applying the LFAC to the nerve with a circumferential (i.e., cuff style) electrode, the fascicles disposed closest to the electrodes will experience the greatest current densities and therefore the greatest changes in polarization. The “spatial recruitment order” of the fascicle fibers having the LFAC applied is referred to as “outside-in.” That is, fascicle fibers nearest the periphery of the nerve may be most affected by the application of the LFAC while more interior fascicle fibers may be less affected. This may not be desirable if a fascicle fiber disposed closer to a center of the nerve is desired to be treated.

Accordingly, there is a need to provide a system and method of nerve therapy that allows blocking and/or activation of fascicles closer to the center of the nerve.

### SUMMARY

In concordance with the instant disclosure, an improved system and technique of nerve therapy, that allows treatment of nerve fascicles near the center of the nerve, has surprisingly been discovered. The present technology includes articles of manufacture, systems, and processes that relate to nerve treatment in animals.

In certain embodiments, a nerve therapy system includes a plurality of electrodes configured to be positioned adjacent a nerve. The system also includes a signal generator electrically connected to the electrodes and configured to generate a low frequency alternating current (“LFAC”) and apply the LFAC to the electrodes. The system further includes a pulse generator electrically connected to the electrodes and configured to generate a pulse current and apply the pulse current to the electrodes simultaneously with the LFAC.

In certain embodiments, a method of nerve therapy includes positioning a plurality of electrodes adjacent a nerve. The method further includes generating a low frequency alternating current (“LFAC”) and applying the LFAC to the electrodes. The method also includes generating a pulse current and applying the pulse current to the electrodes simultaneously with the LFAC.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an electrical schematic of a nerve therapy system according to an exemplary embodiment;

FIG. 2 is a cross-sectional representation of two electrodes situated in a cuff surrounding a nerve according to an exemplary embodiment;

FIG. 3 is a graphical representation of an LFAC waveform combined with a single current pulse at the peak of the LFAC according to an exemplary embodiment;

FIG. 4 is a graphical representation of an LFAC waveform combined with a current pulse at the peak of the LFAC and the trough of the LFAC according to an exemplary embodiment;

FIG. 5 is a graphical representation of an LFAC waveform combined with multiple current pulses according to an exemplary embodiment;

FIG. 6 is an electrical schematic of a nerve therapy system according to an exemplary embodiment;

FIG. 7 is a cross-sectional representation of three electrodes situated in a cuff surrounding a nerve according to an exemplary embodiment;

FIG. 8 is a flow chart showing a method of nerve therapy according to an exemplary embodiment;

FIG. 9 is a graph showing the relationship between LFAC strength and peak pulse current for various sizes of fascicles at a periphery of a nerve;

FIG. 10 is a graph showing the relationship between LFAC strength and peak pulse current for various sizes of fascicles at a depth of 1/4 of the radius of the nerve;

FIG. 11 is a graph showing the relationship between LFAC strength and peak pulse current for various sizes of fascicles at a depth of 1/2 of the radius of the nerve;

FIG. 12 is a graph showing the relationship between LFAC strength and peak pulse current for various sizes of fascicles at a depth of 3/4 of the radius of the nerve; and

FIG. 13 is a graph showing the relationship between LFAC strength and peak pulse current for various sizes of fascicles at a center of the nerve.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology relates to a nerve therapy system 100 and nerve therapy method 800, as shown generally in the accompanying figures.

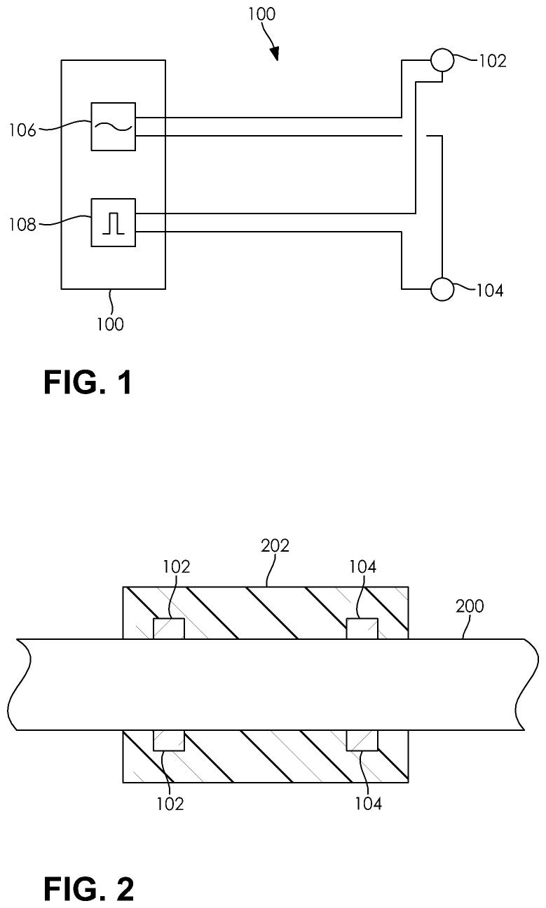

Referring to FIG. 1, the nerve therapy system 100 according to one exemplary embodiment may include a plurality of electrodes 102, 104. In this embodiment, the plurality of electrodes 102, 104 may include a first electrode 102 and a second electrode 104. The electrodes 102, 104 may be configured to be positioned adjacent a nerve 200, as shown in FIG. 2. The electrodes 102, 104 may be in contact with at least a portion of the nerve 200, as appreciated by those of ordinary skill in the art. In this exemplary embodiment, the electrodes 102, 104 may be disposed in a cuff 202. The cuff 202 may extend circumferentially around the nerve 200. The electrodes 102, 104 may also extend circumferentially around the nerve 200.

Numerous manufacturers of such electrode cuffs 202 for use in nerve stimulation and/or sensing are known to those of skilled in the art. One such manufacturer is CorTec GmbH, headquartered in Freiburg, Germany. Of course, other types of electrode packaging, besides a cuff, may be utilized, as is appreciated by those of ordinary skill in the art of nerve stimulation and/or sensing.

Referring again to FIG. 1, the system 100 may also include a signal generator 106 electrically connected to the electrodes 102, 104. The signal generator 106 may be configured to generate a low frequency alternating current (“LFAC”) and apply the LFAC to the electrodes 102, 104. As such, the LFAC may be applied to the nerve 200 and its constituent nerve fibers (not shown) and fascicles (not shown).

The signal generator and the pulse generator may be integrated together into a single power supply unit 110, as shown in FIG. 1. Those of ordinary skill in the art will appreciate numerous modules, techniques, and/or apparatuses to achieve generation and application of the LFAC and pulse currents. In the illustrated embodiments, the LFAC may include a sinusoidal waveform. However, it is possible to utilize other waveforms for the LFAC, including, for example, a triangle waveform or a sawtooth waveform. The LFAC may have a frequency from 0.01 Hz to 100 Hz. The frequency may be varied or may be constant. The LFAC may also have a peak current of ±500 μA. That is, the peak current may oscillate between +500 μA and −500 μA. The peak current may also be varied or may be regular, depending on the application.

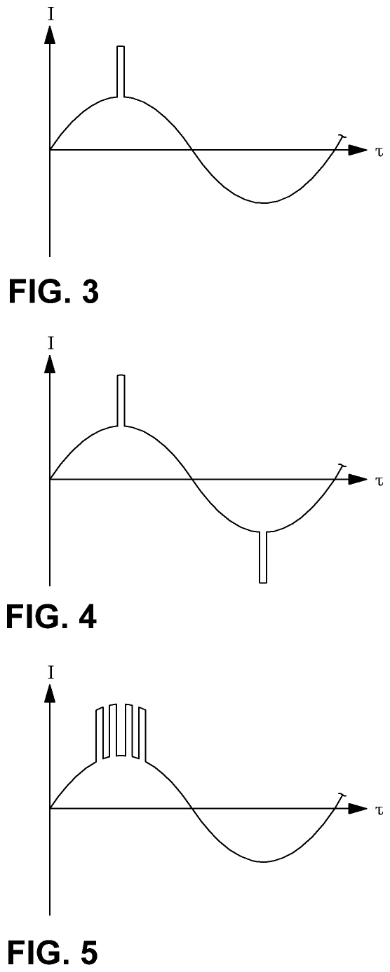

The system 100 may include a pulse generator 108 electrically connected to the electrodes 102, 104. The pulse generator 108 may be configured to generate a pulse current and may apply the pulse current to the electrodes 102, 104 simultaneously with the LFAC. That is, both the LFAC and the pulse current may be applied at the same time, such that pulses may interrupt the regular LFAC waveform. Examples of such a simultaneous application of the LFAC and the pulse current can be seen in FIGS. 3-5. In some applications, the pulse generator 108 may generate a single pulse during each cycle of the LFAC, as is shown in FIG. 3. In other applications, the pulse generator 108 may generate multiple pulses during each cycle of the LFAC, as shown in FIGS. 4 and 5. In some embodiments, the duration of the pulse current may be from 0.1 ms to 10 ms. The duration may be regular or variable in application. For example, the duration of the pulse current may be the same for every pulse or may be different depending on the particular application. In some embodiments, the pulse current may have a maximum current of ±0.1 mA. That is, the pulse current range could be from +0.1 mA to −0.1 mA. For example, as shown in FIG. 4, a positive pulse current may be applied at the positive peak of the LFAC and a negative pulse current is applied at the negative peak of the LFAC.

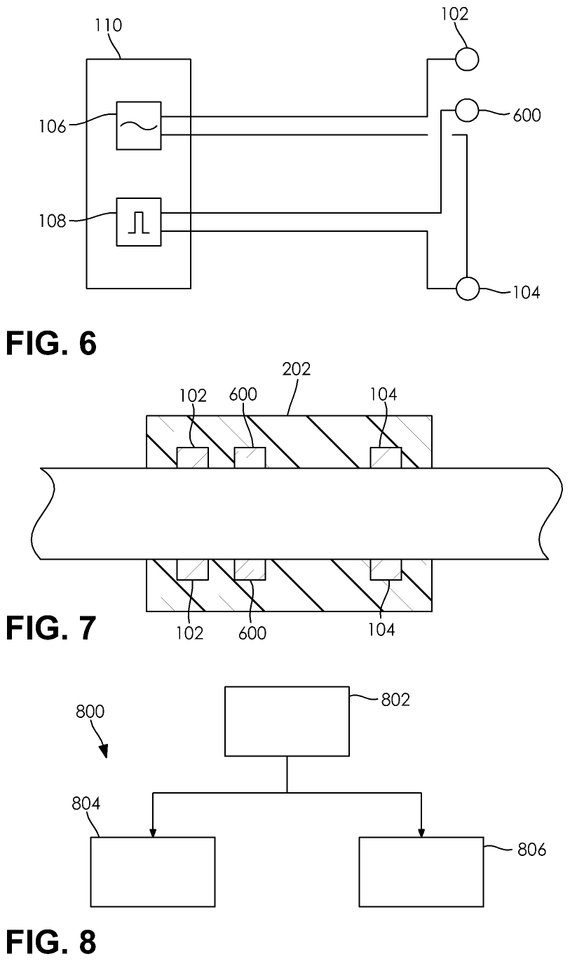

Referring now to FIGS. 6 and 7, the plurality of electrodes 102, 104, 600 may be implemented as the first electrode 102, the second electrode 104, and a third electrode 600. The third electrode 600 may be disposed adjacent the first electrode 102. The first electrode 102 may be electrically connected to the signal generator 106 while the third electrode 600 may be electrically connected to the pulse generator 108. The second electrode 104 may be electrically connected to both the signal generator 106 and the pulse generator 108. In this implementation, current from the signal generator 106 may be delivered to the nerve 200 via the first electrode 102, current from the pulse generator 108 may be delivered to the nerve 200 via the third electrode 600, and current may be returned to the generators 106, 108 via the second electrode 104.

It should be appreciated that the embodiment of the system 100 shown in FIGS. 6 and 7 may produce nearly identical electrical characteristics to the nerve 200 as the system 100 shown in FIGS. 1 and 2. The use of separate electrodes 102, 600 to deliver the pulse current and the signal current may be utilized for convenience when the pulse generator 108 and signal generator 106 are separate devices.

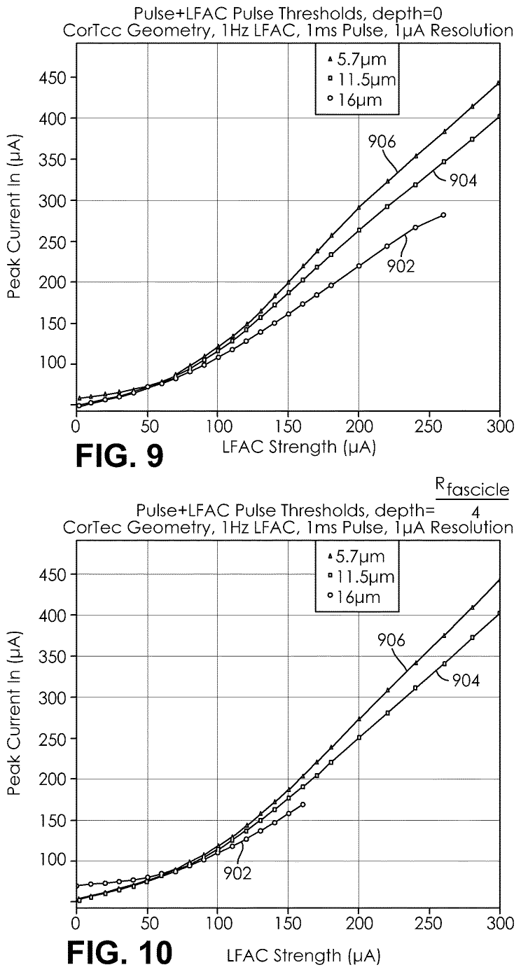

A method 800 of nerve therapy is also disclosed and represented in FIG. 8. It should be appreciated that the method 800 may be implemented using the system 100 described above. Alternatively, the method 800 may be implemented using a different system and/or approach, as will be appreciated by those of ordinary skill in the art. The method 800 includes, at 802, positioning a plurality of electrodes adjacent a nerve. The positioning of the electrodes may be done with any one of numerous surgical techniques familiar to those of ordinary skill in the art. The method 800 also includes, at 804, generating a low frequency alternating current (“LFAC”) and applying the LFAC to the electrodes. The method 800 further includes, at 806, generating a pulse current and applying the pulse current to the electrodes simultaneously with the LFAC. The method 800 may be practiced with the LFAC being sinusoidal, the LFAC having a frequency from 0.01 Hz to 100 Hz, and the LFAC having a peak current of ±500 μA.

The method 800 may also be practiced with the duration of the pulse current being from 0.1 ms to 10 ms and the pulse current having a maximum current of ±0.1 mA. Generating the pulse current may include generating a single pulse current during each cycle of the LFAC or generating multiple pulses during each cycle of the LFAC.

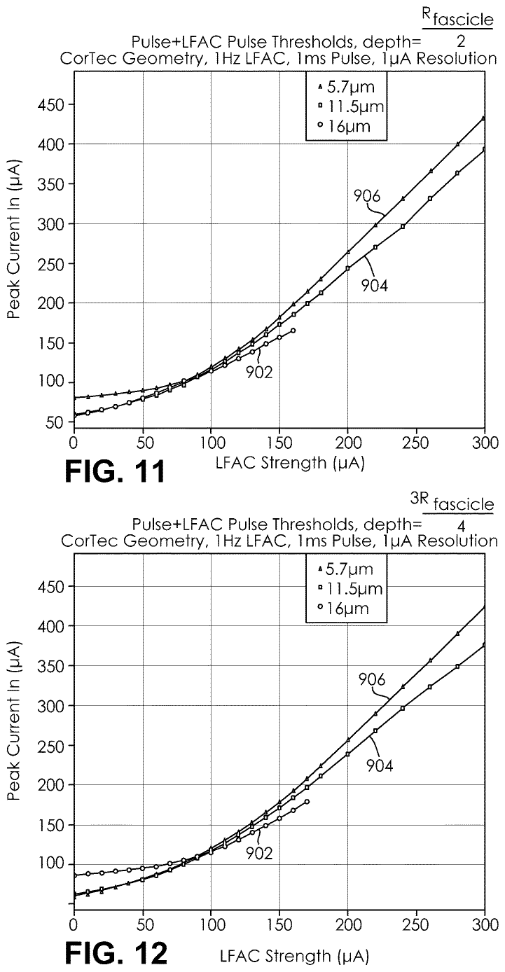

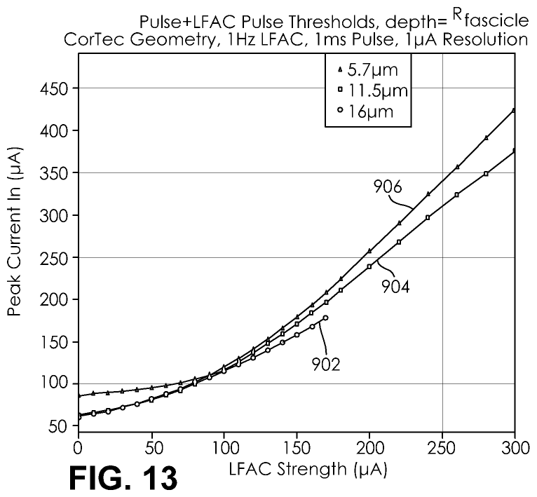

Simulation results of the simultaneous application of the LFAC and the pulse current can be seen with reference to FIGS. 9-13. In these examples, a cuff with two circumferential electrodes is utilized. The electrodes have a cross-sectional width of 0.5 mm and are spaced 1.0 mm apart between conductive edges. A sinusoidal LFAC is applied at 1 Hz. A 1 ms pulse is applied simultaneously at the peaks of the LFAC, such as the example shown in FIG. 4.

The graphs shown in FIGS. 9-13 illustrate the total current applied by the LFAC on the horizontal axis and the total current applied by the pulse current on the vertical axis. The Curve 902 illustrates the total current applied to a 16 μm fascicle fiber, curve 904 illustrates the total current applied to a 11.5 μm fascicle fiber, and curve 906 illustrates the total current applied to a 5.7 μm fascicle fiber. The graphs show how the depth order inverts at LFAC block thresholds. Prior to this inversion, the fascicle fibers experienced a relative sensitization. That is, the absolute contribution of the pulse was reduced with increasing LFAC strength. This inverted order was more strongly present for larger fibers. It has also been discovered that the combined waveform also converts the fiber diameter recruitment order to that of the body's natural order. Pulse on its own is known to activate large nerve fibers first, however when applied on top of sufficiently strong LFAC the order inverts to activating small fibers first, as can be seen in FIGS. 9-13, which is the natural order in which the body recruits these fibers and results in less muscle fatigue.

Described embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A therapy system for a nerve, comprising:
a plurality of electrodes configured to be positioned adjacent the nerve;
a signal generator electrically connected to the electrodes and configured to generate a low frequency alternating current (LFAC) and apply the LFAC to the electrodes; and
a pulse generator electrically connected to the electrodes and configured to generate a pulse current and apply the pulse current to the electrodes simultaneously with the LFAC.

2. The therapy system of claim 1, wherein the LFAC has a frequency from 0.01 Hz to 100 Hz.

3. The therapy system of claim 1, wherein the LFAC has a peak current of ±500 μA.

4. The therapy system of claim 1, wherein the LFAC includes a sinusoidal waveform.

5. The therapy system of claim 1, wherein the duration of the pulse current is from 0.1 ms to 10 ms.

6. The therapy system of claim 1, wherein the pulse current has a maximum current of ±0.1 mA.

7. The therapy system of claim 1, wherein the pulse generator is configured to generate a plurality of pulse currents.

8. The therapy system of claim 1, wherein the plurality of electrodes includes a first electrode and a second electrode, each of the first electrode and the second electrode electrically connected to the signal generator and the pulse generator.

9. The therapy system of claim 1, wherein the plurality of electrodes includes a first electrode, a second electrode, and a third electrode disposed adjacent the first electrode, the first electrode electrically connected to the signal generator, the second electrode electrically connected to the signal generator and the pulse generator, and the third electrode electrically connected to the pulse generator.

10. The therapy system of claim 1, wherein each electrode of the plurality of electrodes is disposed in a cuff configured to extend circumferentially around the nerve.

11. The therapy system of claim 1, wherein the signal generator and the pulse generator are integrated together in a power supply unit.

12. A therapy system for a nerve, comprising:
a plurality of electrodes configured to be positioned adjacent the nerve;
a signal generator electrically connected to the electrodes and configured to generate a low frequency alternating current (LFAC) and apply the LFAC to the electrodes; and
a pulse generator electrically connected to the electrodes and configured to generate a pulse current and apply the pulse current to the electrodes simultaneously with the LFAC;
wherein:
the LFAC has a frequency from 0.01 Hz to 100 Hz;
the LFAC has a peak current of ±500 μA;
the LFAC is sinusoidal;
the duration of the pulse current is from 0.1 ms to 10 ms;
the pulse current has a maximum current of ±0.1 mA;
the pulse generator generates a plurality of pulse currents during each cycle of the LFAC;
the plurality of electrodes includes a first electrode, a second electrode, and a third electrode disposed adjacent the first electrode, wherein the first electrode is electrically connected to the signal generator, the second electrode is electrically connected to the signal generator and the pulse generator, and the third electrode is electrically connected to the pulse generator;
each electrode of the plurality of electrodes is disposed in a cuff configured to extend circumferentially around the nerve; and
the signal generator and the pulse generator are integrated together in a power supply unit.

13. A method of therapy for a nerve, comprising:
providing a therapy system, including
a plurality of electrodes configured to be positioned adjacent the nerve;
a signal generator electrically connected to the electrodes and configured to generate a low frequency alternating current (LFAC) and apply the LFAC to the electrodes; and
a pulse generator electrically connected to the electrodes and configured to generate a pulse current and apply the pulse current to the electrodes simultaneously with the LFAC;
positioning the plurality of electrodes adjacent the nerve;
using the signal generator to generate the low frequency alternating current (LFAC) and applying the LFAC to the electrodes; and
using the pulse generator to generate the pulse current and applying the pulse current to the electrodes simultaneously with the LFAC.

14. The method of claim 13, wherein the LFAC has a frequency from 0.01 Hz to 100 Hz.

15. The method of claim 13, wherein the LFAC has a peak current of ±500 μA.

16. The method of claim 13, wherein the LFAC is sinusoidal.

17. The method of claim 13, wherein the duration of the pulse current is from 0.1 ms to 10 ms.

18. The method of claim 13, wherein the pulse current has a maximum current of ±0.1 mA.

19. The method of claim 13, wherein using the pulse generator to generate the pulse current comprises generating a plurality of pulse currents during a plurality of cycles of the LFAC.
